(19) United States
(12) Patent Application Publication
(10) Pub. No.: US 2006/0045924 A1
(43) Pub. Date: Mar. 2, 2006

(54) Dried fruit composition with laxative effects and method for administration thereof

(76) Inventors: Lily Chen, Granada Hills, CA (US); Maggie Yea, Arcadia, CA (US)

Correspondence Address: Lily Chen, PO Box 162667, Sacramento, CA, 95816, US

(21) Appl. No.: 11/212,941

(22) Filed: Aug. 25, 2005

Related U.S. Application Data
(60) Provisional application No. 60/606,460, filed on Aug. 31, 2004.

(51) Int. Cl. A61K 36/736; A61K 36/752

(52) U.S. Cl. 424/735; 424/777; 424/736; 424/765

(57) ABSTRACT

A dried fruit composition enhanced with herbal extracts and a healthful dose of bacteria for enhanced laxative effects and appetite suppression. Preferred methods of manufacture and methods for oral administration of the dried fruit composition are further disclosed.

### RELATED APPLICATIONS

This application claims priority to Provisional Application 60/606,460 filed on Aug. 31, 2004.

### FIELD OF THE INVENTION

The present invention relates to dietary supplements and foods for improving the human digestive system's functions, including relieving constipation, treating hemorrhoids, and controlling weight gain. More specifically, the invention relates to the field of natural compositions consisting of dried fruit and herbal extracts treated with effective doses of healthful bacteria, and oral administration of such compositions to improve digestive metabolism.

### BACKGROUND OF THE INVENTION

Constipation is a common digestive problem in human beings, and usually occurs due to abnormal motility patterns in the colon leading to delayed propulsion of the intestinal contents. It also occurs due to weak peristaltic movements or defecation reflex leading to hindrance in propagation of the stool.

Constipation is also incident to high-protein, low-carbohydrate diets commonly used to achieve weight loss, since such low-carbohydrate diets are necessarily low in fiber.

Common laxatives used to treat constipation include synthetic chemical compounds such as phenolphalein, bisacodyl (diphenyl methane laxatives), milk of magnesia and the like.

These laxatives, although effective, have been found to exhibit undesirable side effects, the most common of which are abdominal pain, cramps, nausea, skin rash, intestinal, and rectal bleeding. High intake of such bulk laxatives also sometimes results in undesirable addiction to these products, leading to even more severe constipation when use of the products ceases.

Furthermore, many of the commercial laxatives fail to be incorporated into a normal person's diet due to their unappealing taste. Natural fruits and vegetables that seem to have good laxative action, including citrus fruits, rhubarb, spices, cabbage, prunes, figs and garlic, offer more appealing taste at the expense of somewhat reduced laxative efficacy.

It is thus the primary object of the present invention to provide a dietary supplement in the form of a nutritional snack that offers enhanced laxative effect compared to natural fruits and vegetables, preserving and even enhancing delicious taste while simultaneously foregoing the negative side-effects of strong chemical laxatives.

It is a further object to provide a composition that relieves constipation while helping diet-conscious users control their weight through natural appetite suppression, while avoiding the symptoms of constipation associated with high-protein, low-carbohydrate diets commonly used to achieve weight loss.

It is yet another object of this invention to provide a method of processing individual ingredients to make a composition having such beneficial qualities palatable and delicious to the user, to induce long-term and continuous use thereof as a dietary supplement.

It is yet another object of this invention to offer the benefits of the said composition in a small and convenient package.

Yet another object of this invention is to prescribe a method whereby users can orally ingest said composition according to a daily regimen, and vary their intake of said composition to optimally achieve different desired effects on the digestive system.

### DESCRIPTION OF THE RELATED Art

The related art of interest describes various laxatives and/or diets to control weight gain, but none discloses the present invention. There is a need for a delicious snack that will provide enhanced laxative effects, but also curb one's appetite to prevent overeating. The related art will be discussed in the order of perceived relevance to the present invention.

U.S. Pat. No. 6,641,851 issued on Nov. 4, 2003, to Mitra, et al. describes an herbal laxative preparation based on chloroform or methanolic extraction of rhizomes and/or roots of Picrorrhiza Kurroa. The composition is distinguishable in its ingredients, and also for its lack of mention of the taste of the composition as an important factor. It also does not have any appetite suppressant capability.

U.S. Pat. No. 6,641,808 issued on Nov. 4, 2003, to Bojrab describes a composition for treatment of obesity comprising a culture having Lactobacillus bulgaricus and Streptococcus thermophilus lactic acid bacteria and a carbohydrate enriched media. The composition is distinguishable in its ingredients, which does not mention dried fruit as the agent of delivery of Lactobacillus bacteria, nor the herbal extracts, nor the taste of the overall composition that are disclosed herein. U.S. Pat. No. 6,573,083 issued on Jun. 3, 2003, to Raider describes a method of weight reduction in human beings, wherein a human ingests a laxative effective dosage of non-pathogenic Escherichia coli. The method is distinguishable from the current invention, as the current invention utilizes Lactobacillus bacteria rather than E. coli. Furthermore, the patented method does not mention the taste, nor appetite suppressant effects inherent in the present invention.

U.S. Pat. No. 6,541,046 issued on Apr. 1, 2003, to Wu, et al. describes an herbal composition and method for controlling body weight and composition, disclosing a decoction of a herbal mixture, comprising rhubarb, red saga root, astragalus, turmeric, and dried ginger and various combinations thereof, provides therapeutic weight loss as well as lipid reduction and change body composition. The composition is distinguishable from the current invention in the ingredients, as well as the lack of mention of taste as well as appetite suppressant effects inherent in the present invention.

### SUMMARY OF THE INVENTION

The present invention provides a dietary supplement in the form of a nutritional snack that offers enhanced laxative effect compared to natural fruits and vegetables, foregoing the negative side-effects of strong chemical laxatives while providing delicious taste. The invention is comprised of a new composition and method for improving intestinal health by relieving constipation and hemorrhoids, controlling weight gain, and improving the overall function of the digestive system. The composition's form as a delicious snack, combined with its stool-softening and natural appetite-suppressing effects, promotes one's appetite, and improves the health of the digestive system. The composition's active ingredients act to soften the contents of the digestive tract, thereby promoting quick excretion of stool to prevent the absorption of calories, while also suppressing the user's appetite. The composition is packaged in the form of a small piece of delicious dried fruit, which facilitates ingestion by the user as a snack before, during, or after meals.

The composition comprises dried fruit, herbal extracts, and an effective dosage of healthful bacteria. The composition is made of all-natural ingredients, and does not have any of the harmful side effects associated with traditional laxatives or weight-loss compounds. In a preferred embodiment, the composition includes as ingredients a dried golden plum, mixed with herbal extracts including Ear-type leguminosae and Angelica keiskei (available from Chinese medicine supply stores), as well as an effective dose of Lactobacillus acidophilus bacteria. In a preferred embodiment, the ingredients are processed according to a series of steps including drying the fruit, combining with the mixture of herbal extracts and healthful bacteria, baking in an oven to seal in the ingredients, and irradiating with ultraviolet rays to prevent contamination from unwanted bacteria.

In addition, a method for ingesting the new composition according to a scheduled regime, that may be optimally tailored to each individual user's health needs, is recited herein.

### DETAILED DESCRIPTION OF THE INVENTION

I. The Composition

The new composition is comprised of dried fruit, herbal extracts and a dosage of healthful bacteria. Together, these ingredients are effective in regulating the digestive system. The specific ingredients of the composition are disclosed herein.

I.A. The Dried Fruit

The fruit used for the dried fruit can be any of typical fruits including, but not limited to, all species of plums, mango, peach, olive, banana, apple, cherry, and Carambola. In the preferred embodiment, the fruit is a Japanese golden plum, also known as “umeboshi,” or Japanese apricot. So that the fruit retains its taste and is amenable to preservation for extended periods of time, it is dried using standard fruit-drying processes well-known in the art. For a preferred embodiment of a drying process, see Section II.

I.B. The Herbal Extracts

The herbal extracts are naturally-occurring ingredients which, when taken in combination, exhibit two effects: one effect is to soften the stool within the digestive tract and thereby improve bowel movement leading to ready excretion of the digestive tract contents; another effect is to suppress the appetite to reduce the desire for further intake of food after the induced excretions.

In a preferred embodiment of the invention, the herbally extracted ingredients for softening the stool are comprised of:
HCA (garcinia cambogia), a compound with known appetite suppressant qualities.
Angelica keiskei
S. Auricula
Puu Eel Tea
Cangzhu Atractylodea
Chenpi Criad Tangerine Peel
Catechines
Plum Powder

Each ingredient is separately ground into a fine powder, and mixed with other ingredients. The resultant mixture is also of a powder form. In a preferred embodiment, each ingredient is present in equal proportion by mass to every other ingredient. For example, 10 grams of plum powder will be mixed with 10 grams of catechines, mixed with 10 grams of Chenpi Criad Tangerine Peel, etc. The mixture is the herbal extract aspect of the present invention, and can be combined with the dried fruit and bacteria parts of the composition according to the process disclosed in Section II.

All of the above ingredients are readily obtainable from stores that specialize in supplying Chinese medicine ingredients. The amounts of these herbal extracts present in the composition of the invention are such that the overall composition, when ingested, induces the desired effects of improving bowel movement and suppressing appetite. In a preferred embodiment, the inventor has found that using enough powder to evenly coat a piece of dried plum fruit by rolling the fruit about two times in the powder mixture suffices for the purposes of the present invention.

One who is ordinarily skilled in the art of herbal remedies will recognize that the aforementioned ingredients may be present in proportions slightly more or less than the equal proportions cited above, while still retaining their potency to induce the desired effects. In fact, one skilled in the art will recognize that the absence of one or more of these ingredients in the composition may still induce the desired effect, as long as other ingredients are present in sufficient quantity. Moreover, one skilled in the art will also recognize that other naturally-occurring herbal ingredients and extracts thereof which mimic the effects of the ingredients described herein may be freely substituted without detracting from the spirit and utility of the present invention.

I.C. The Healthful Dose of Bacteria The healthful bacteria used in this invention are especially selected to induce desirable laxative and appetite suppressant effects, and operate in conjunction with the dried fruit described in Part I.A, and the Chinese medicine ingredients described in Part I.B. The bacteria are selected from the group comprising Streptococus lactis, Enterococcus faecium, Lactobacillus acidophilus, Bifidobacterium bifidum, Streptococcus thermophilus, and mixtures thereof. In a preferred embodiment, the dosage includes each of these types of bacteria in equal proportion by mass.

The following is a description of a preferred method for obtaining the bacteria from starter cultures.
1) Prepare a mixture of milk (any type of milk may be used) and fruit juices (preferably juice from Lomeng fruit). Any milk or juice that can support the bacterial strains will work.
2) Heat the mixture to above about 110 degrees C. to kill undesirable contaminant microorganisms.
3) Cool the mixture to about 25-33 degrees C.
4) Add starter culture comprising one strain of the healthful dose of bacteria described above into the mixture. For each preparation, each strain of bacteria should be prepared separately, as the strains generally have different growth rates.
5) Incubate the mixture at 25-33 degrees C. for about 24-48 hours.
6) Cool and dry at about 5 degrees C. The mixture will condense into a white powder.
7) Repeat steps 1)-6) for each strain of bacteria.
8) Mix the bacteria in white powder form in approximately equal proportions to obtain the powder to be mixed with the dried fruit.

Note as in the preparation of the herbal extracts in Section I.B, one who is ordinarily skilled in the art of remedies will recognize that the aforementioned bacterial strains may be present in proportions slightly more or less than the equal proportions cited above, while still retaining their potency to induce the desired effects. In fact, one skilled in the art will recognize that the absence of one or more of these strains in the composition may still induce the desired effect, as long as other ingredients are present in sufficient quantity. Moreover, one skilled in the art will also recognize that other naturally-occurring herbal ingredients and extracts thereof which mimic the effects of the ingredients described herein may be freely substituted without detracting from the spirit and utility of the present invention.

II. Process of assembling the composition

The above ingredients occur naturally, and may simply be mixed together without further processing to obtain an ingestible snack with the desired beneficial effects on the digestive system. Such an assembly of the raw ingredients falls within the scope of the present invention. However, further experimentation has been done to optimize the composition's taste and maximize the preservability and shelf-life of the composition, leading to a preferred embodiment in which the cited ingredients of the invention are processed as described below.
1. Select fresh golden plums, wash, then peel skin, then let dry. Note the plums do not need to be pitted.
2. Pre-treat plums with salt, alcohol, and sulfur dioxide for about 60 minutes (preferably in a porcelain jar), for antioxidant effects. The sulfur preserves the color of the plum, as well as helps prevent loss of vitamins A and C. The sulfur is not a preservative in itself, but it discourages insects and microbes which can spoil the fruit.
3. Mix pre-treated plums with sugar (e.g., sugarcane sugar or sucrose) and sulfite preservatives, in sufficient quantity to cover the pre-treated plums. Let the plum sit for about 90 days. The result of this drying process is known as the plum base.
4. Add the herbal extracts described in Part I.B to plum base. Optionally, additional flavoring can be mixed in for a sweet and sour taste. This flavoring can be sugar mixed with some alcohol. Mix extracts and flavoring together with plum, and let it sit for about 48 hours to lock ingredients into plum. The more time the plum is allowed to mix with extracts and flavoring, the greater the effect of the extracts and the richer the flavoring.
5. Heat for 120-150 mins at 120° C. to kill germs.
6. After allowing to cool, then add bacterial ingredients from Section I.B. in the form of powder. Evenly coat plums with powder. To do this, place the dried fruit in a sufficiently large jar full of the mixed powder and keep the fruit wholly immersed for about 48 hours. This allows the mixture to evenly coat the dried fruit and seep into it. The more time is allowed, the more effective will be the dosage of bacteria.
7. Cool with UV/ozone system to prevent contamination from unwanted germs (e.g., at approximately 5 degrees C.). Vacuum-package individually treated pieces of dried plums using standard nitrogen packaging unit to protect composition from ambient oxygen, humidity and oxidation problems. This packaging extends shelf-life.
8. For extra protection from contaminant bacteria, further vacuum package the individual packages into a 460-gram sealed bag, or other suitably sized bag, using the same nitrogen techniques as in Step 7. The collective package will thus contain many individually packaged plums. (Note that because individual plum weights may vary, the number of actual plums in a specific 460-gram collective package will vary.)

III. Method of administering the composition for optimal effectiveness For optimal effect, the composition is to be ingested orally on a regular basis. In one embodiment of the method for administering the composition, the composition is ingested after meals, e.g., dinner. After ingestion, drink about 300 cc of water. Since the composition promotes quick excretion of the digestive tract contents upon ingestion, up until the time its active ingredients are exhausted, the period of time that elapses between completion of the meal and ingestion of the composition can be varied to achieve different desired effects. In one embodiment, ingestion of the composition immediately after a meal prevents absorption of the fat and calories of the meal, and further suppresses the appetite, leading to control of weight gain. In another embodiment of the invention, ingestion of the compound 1-2 hours after a meal allows time for the nutritional contents of the meal to be digested, and promotes excretion of the stool, thereby leading to relief of constipation.

In another embodiment, the composition is orally administered prior to or during meals, to promote healthy bowel movement during digestion of the meal proper. The appetite suppressant effects further control food consumption during the meal leading to weight control.

In a further embodiment, the composition may be orally administered as a stand-alone snack.

Note the effective dosage of the composition will depend on the user's body weight and metabolism. For example, informal trials suggest that one standard dose per day of a dried plum embodiment of the invention, consisting of one dried plum processed according to Section II, is effective in relieving constipation and noticeably suppressing appetite in a 150-pound adult male.

Note that since the composition does not include any artificial chemicals or laxatives, consumption in large quantities will not be deleterious to the health, but will simply promote frequent trips to the restroom. When the contents of the stomach and intestines have been purged through excretion, the active ingredients of the present composition cease to have effect, and will not induce diarrhea or dehydration.

The previous description of the preferred embodiments is provided to enable any person skilled in the art to make or use the present invention. The various modifications to these embodiments will be readily apparent to those skilled in the art, and the generic principles defined herein may be applied to other embodiments without the use of the inventive faculty. Thus, the present invention is not intended to be limited to the embodiments shown herein but is to be accorded the widest scope consistent with the principles and novel features disclosed herein.

What is claimed is:

1. A dried fruit composition comprising:
A dried fruit;
Herbal extract, present in sufficient quantity to induce bowel movement and suppress appetite; and
Powdered bacteria, present in sufficient quantity to induce bowel movement.

2. The composition of claim 1, wherein the dried fruit is selected from the group consisting of:
Plums, mango, peach, olive, banana, apple, cherry, Carambola, and Japanese golden plum.

3. The composition of claim 2, wherein the dried fruit is Japanese golden plum.

4. The composition of claim 1, wherein the herbal extract is selected from the group consisting of:
HCA (garcinia cambogia), Angelica keiskei, S. Auricula, Puu Eel Tea, Cangzhu Atratylodea, Chenpi Criad Tangerine Peel, Catechines, and Plum Powder.

5. The composition of claim 4, wherein the herbal extract comprises:
HCA (garcinia cambogia), Angelica keiskei, S. Auricula, Puu Eel Tea, Cangzhu Atratylodea, Chenpi Criad Tangerine Peel, Catechines, and Plum Powder.

6. The composition of claim 1, wherein the powered bacteria is selected from the group consisting of:
Streptococus lactis, Enterococcus faecium, Lactobacillus acidophilus, Bifidobacterium bifidum, and Streptococcus thermophilus.

7. The composition of claim 6, wherein the powdered bacteria comprises:
Streptococus lactis, Enterococcus faecium, Lactobacillus acidophilus, Bifidobacterium bifidum, and Streptococcus thermophilus.

8. The composition of claim 1, wherein:
The dried fruit is Japanese golden plum;
The herbal extract comprises HCA (garcinia cambogia), Angelica keiskei, S. Auricula, Puu Eel Tea, Cangzhu Atratylodea, Chenpi Criad Tangerine Peel, Catechines, and Plum Powder; and
The powdered bacteria comprises Streptococus lactis, Enterococcus faecium, Lactobacillus acidophilus, Bifidobacterium bifidum, and Streptococcus thermophilus.

9. The composition of claim 1, wherein:
The herbal extract is present in an amount corresponding to evenly coating the dried fruit in the powdered herbal extract by rolling it in the herbal extract about two times.

10. The composition of claim 1, wherein:
The powdered bacteria is present in an amount corresponding to evenly coating the dried fruit in the powdered bacteria by wholly immersing the dried fruit in the powdered bacteria for about 48 hours.

11. The composition of claim 1, wherein:
The fruit is first peeled, then dried;
The dried fruit is treated with salt, alcohol, and sulfur dioxide for about 60 minutes;
The treated dried fruit is mixed with sugar and sulfite preservatives in sufficient quantity to cover said dried fruit for a period of about 90 days;
The treated fruit is further mixed with the herbal extract for about 48 hours;
The fruit is then heated at approximately 120° C. for approximately 120-150 minutes;
The heated fruit is allowed to cool;
The cooled fruit is immersed in and coated with the powered bacteria means for about 48 hours;
The fruit is then treated with a UV or ozone system.

12. A method for ingesting the composition of claim 1, comprising the steps of:
Orally ingesting the composition up to two hours before, or up to two hours after, or during meals; and
Drinking water immediately after orally ingesting the composition.

13. The method of claim 12, wherein the composition is orally ingested about 1-2 hours after meals.
